(19) United States
(12) Patent Application Publication
(10) Pub. No.: US 2008/0206834 A1
(43) Pub. Date: Aug. 28, 2008

(54) RECOMBINANT ORGANOPHOSPHOROUS ACID ANHYDRASE AND METHODS OF USE

(76) Inventors: C. STEVEN MCDANIEL, HOUSTON, TX (US); FRANK M. RAUSHEL, COLLEGE STATION, TX (US); JAMES R. WILD, COLLEGE STATION, TX (US)

Correspondence Address: DAFFER MCDANIEL, LLP, P.O. BOX 684908, AUSTIN, TX, 78768-4908, US

(21) Appl. No.: 08/252,384

(22) Filed: Jun. 1, 1994

Related U.S. Application Data
(63) Continuation of application No. 07/928,540, filed on Aug. 13, 1992, now abandoned.
Application No. 07/928,540 is a division of application No. 07/344,258, filed on Apr. 27, 1989, now abandoned.

(51) Int. Cl. C12N 9/16; C07H 21/04; C12N 1/21; C12N 5/06; C12N 15/09; C12N 15/74

(52) U.S. Cl. 435/196; 435/325; 435/252.1; 435/320.1; 536/23.2; 435/455; 435/471

(57) ABSTRACT

The bacterial gene (opd) encodes an organophosphorus anhydrase which is capable of hydrolyzing a wide spectrum of neurotoxins ranging from insecticides to mammalian neurotoxins. The cloned gene has been expressed in a number of hosts and the purified enzyme has been characterized. These advances have led to a number of interrelated uses for the cloned gene and the recombinant enzyme including: detoxification of organophosphorus compounds; detection of organophosphorus compounds; protection of susceptible organisms from organophosphorus poisoning; and, detection of organophosphorus-detoxifying microorganisms.

The U.S. Army Research Office provided funding used in part for this invention under contract 21288-LS (6/84-6/86) and contract DAALO3-87-K-0017 (12/86-12/89). Accordingly, the Federal Government may have certain rights in this invention pursuant to 35 U.S.C. 202.

### BACKGROUND OF THE INVENTION

A. Field of the Invention

This invention relates to genetic engineering. The invention relates particularly to a cloned bacterial gene and heterologous expression of the gene in diverse biological hosts using a variety of expression vectors. The invention relates further to uses of the recombinant enzyme encoded by the bacterial gene. More particularly, the invention relates to the use of the recombinant gene and/or enzyme to detoxify and detect organophosphorus neurotoxins or to detect microorganisms capable of detoxifying organophosphorus compounds.

B. Description of Related Art

Synthetic organophosphorus neurotoxins are used extensively as agricultural and domestic pesticides. Organophosphorus compounds are also common components of nerve gases found in chemical warfare arsenals.

A variety of problems have arisen due to the use of organophosphorus compounds. Chief among these problems is the lack of an effective means for safe and thorough disposal. Currently, organophosphorus compounds are detoxified by either basic hydrolysis, dilution in aqueous solutions or incineration. These techniques are not efficient and may cause environmental pollution. Massive stockpiles of aging nerve gas containers are particularly difficult to detoxify using current methodology.

Another problem arises from the difficulty in protecting personnel, supplies and equipment from potentially hazardous organophosphorus vapors. This is a serious problem under field conditions experienced by military personnel under threat of chemical attack. One means for providing such protection used currently is by enzymatic detoxification using the enzyme DFPase of giant squid axon. However, the availability of DFPase is limited making such protection very expensive.

A similar problem arises when organophosphorus compounds are used to treat crops near beehives. The unprotected insects are susceptible to oversprays which may seriously curtail honey production. Similarly, other beneficial insects such as silk worms can be seriously endangered by use of organophosphorus compounds near silk manufacturing operations or on food plants used in silk manufacture. No adequate means exists for protecting such insect-based operations.

Another set of problems regarding organophosphorus compounds arises out of the necessity of detecting such compounds in a variety of settings. Not the least of these problems involves the detection of potentially hazardous organophosphorus vapors. Means currently in use to detect organophosphorus compounds typically require bulky, sophisticated equipment. The use of such equipment, particularly in field settings, is impractical. Even when such equipment can be used, such as in a laboratory setting, means for rapidly confirming the presence of organophosphorus compounds in trace amounts are required.

Awareness of a unique set of problems associated with the biolabile organophosphorus pesticides has recently arisen. The effectiveness of organophosphorus pesticide applications is compromised by the presence of soil bacteria capable of rapidly detoxifying these pesticides. No means are currently available to detect such bacteria prior to pesticide application. Without a means of testing for bacteria capable of detoxifying organophosphorus pesticides, applications of organophosphorus compounds to crops may be ineffective.

In an effort to resolve some of these problems, naturally occurring bacterial isolates capable of metabolizing the organophosphorus compounds have received considerable attention (1,2) since they provide the possibility of both environmental and in situ detoxification (reviewed in 3). [As used herein, numbers in parentheses refer to references in the bibliography unless indicated otherwise.] Pseudomonas and Flavobacterium species have been shown to possess the ability to degrade an extremely broad spectrum of organophosphorus phosphotriesters as well as thiol esters. However, none of these strains have shown the ability to use neurotoxins as sole nutrient sources. Consequently, selection of strains suitable for recombinant DNA research has been difficult.

Two bacterial strains from the closely related genera Pseudomonas and Flavobacterium have been found to encode (opd) genes on large plasmids (4,5,6). The genetic locations of the degradative genes are unknown in other bacteria (7,8). The isolation and subcloning of opd from these large, degradative plasmids (40 to 65 kilobases in size) has proven to be very difficult (5,6,9). In particular, expression of the opd gene in heterologous backgrounds has not been feasible on a commercial basis.

There have been numerous attempts to characterize the enzyme encoded by the opd gene using crude extracts of the native bacteria (10,11). However, limitations arise using this approach due to two factors. First, these soil bacteria are difficult to culture. Second reliance on crude extracts for the necessary characterization of the enzyme can be misleading and costly. With the increasing use of organophosphorus neurotoxins in modern society, means for detoxifying and detecting such compounds and means for maximizing their effectiveness are needed.

### SUMMARY OF THE INVENTION

The present invention provides an organophosphorus detoxifying (opd) gene of the DNA sequence set forth in FIG. 1; a recombinant bacterial organophosphorus acid anhydrase (OPA) enzyme derived from the opd gene; a collection of expression vectors comprising the opd gene; a collection of transformed cells comprising the opd gene on an expression vector; and, transgenic organisms comprising the opd gene on an expression vector. Furthermore, the invention provides several methods for using the gene, vectors, cells and organisms of the invention, namely: a method for making commercial quantities of OPA; a method for purifying the OPA; methods for using either recombinant opd microorganisms or the purified OPA to detoxify organophosphorus compounds; a method for detecting opd-containing microorganisms; a method for detecting organophosphorus compounds in residue analysis or in air samples; a method for protecting beneficial insects using either recombinant microorganisms or recombinant protein or the opd gene itself; and, a method for co-formulating organophosphorus pesticides prior to application.

### BRIEF DESCRIPTION OF FIGURES

FIG. 1 shows the nucleotide sequence of the opd gene.

FIG. 2 illustrates the activity of certain opd subclones. Sequences are adjusted to align vector DNA on either side of the opd subclone. Deletions are indicated by open space between brackets. The putative coding region for the opd gene is indicated by shading, and the sense direction is shown by arrows.

FIG. 3 shows the construction of the opd expression vector for use in insect cells.

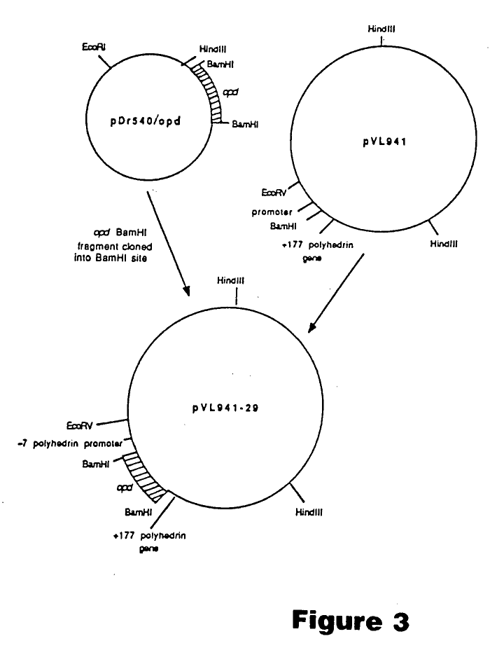

FIG. 4 shows the mortality curve for infected and uninfected caterpillars.

FIG. 5(a) shows the master plate of Pseudomonas diminuta. The arrow indicates a colony subsequently shown to lack OPA activity; FIG. 5(b) is a filter lift of the master plate in FIG. 5(a) which has been allowed to develop 4-nitrophenol coloration and is subsequently photographed using UV-illumination. The arrow indicates the same colony as described in FIG. 5(a); FIG. 5(c) is an image produced by overlapping (eclipsing) FIGS. 5(a) and 5(b). The round, bright colony in the lower right-hand corner of the image corresponds to that described in FIGS. 5(a) and 5(b).

FIG. 6 shows the derivation of plasmids containing opd from Pseudomonas diminuta or Flavobacterium sp. where (a) is the P. diminuta plasmid pCMS1, and (b) is the Flavobacterium sp. plasmid.

FIG. 7 shows a Southern blotting and hybridization of the 1.3-kb probe with plasmid DNAs from P. diminuta and a Flavobacterium sp.

FIG. 8 shows a Southern blotting and hybridization of the 0.9-kb probe with plasmid DNAs from P. diminuta and a Flavobacterium sp.

### DETAILED DESCRIPTION OF THE INVENTION

This invention provides a biological means of detoxifying and detecting organophosphorus compounds. The invention provides as well a means for protecting susceptible organisms from organophosphorus poisoning. The invention also provides means for detecting organophosphorus-detoxifying microorganisms and for co-formulating pesticides for maximal efficiency. In order to achieve these ends, the invention relies on the heterologous expression of the specific DNA sequence containing the organophosphorus detoxifying (opd) gene encoding the recombinant organophosphorus acid anhydrase enzyme (OPA).

### Heterologous Expression of the opd Gene

The opd gene is isolated by first isolating the native plasmid DNA of an organophosphorus-detoxifying bacterium such as Pseudomonas diminuta or Flavobacterium sp. (ATCC 27551). Surprisingly, the inventors discovered that, in at least two cases, these native plasmids carry the identical opd gene (This is further elaborated in Harper, et al. 1988. Appl. Environ. Microbiol. 54:2586-2589). The two DNA sequences are invariant but the native plasmid vectors are different. It is this precise DNA fragment which was isolated by the inventors and which allowed heterologous expression.

The opd gene fragment derived from the native plasmid DNA above is purified by any one of a number of methods known to those of skill in the art and is then inserted into an expression vector chosen for its ability to transform a given cell. Typically, the initial subcloning would be made by transforming the bacterial cell E. coli. The use of a PstI-derived fragment substantially free of extraneous DNA is critical to the success of the initial cloning since the heterologous expression of a Pseudomonas or Flavobacterium gene in E. coil is difficult to achieve. This discovery allowed the inventors to succeed in obtaining heterologous expression where others had failed to do so. (This is explained in McDaniel, et al. 1988. J. Bacteriol. 170:2306-2311).

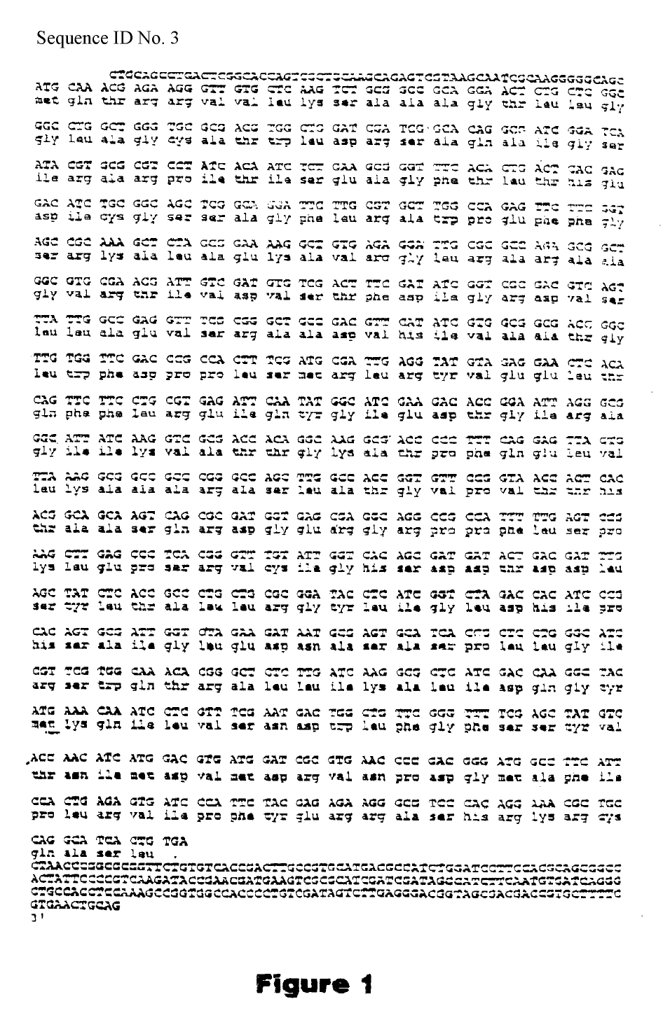

In order to carry out the steps necessary to adequately express the opd gene, it is necessary to determine the sequence of the DNA encoding it. This can be achieved by methods known to those skilled in the art and is illustrated in FIG. 1 for the PstI fragment containing the opd gene.

It is equally important to determine the correct reading frame prior to manipulating the gene in order to increase expression. Determining the correct reading frame can be accomplished by isolating the gene's protein product and amino acid sequencing the protein using techniques known to those skilled in the art. Preferably one isolates a fusion polypeptide the opd portion of which when sequenced confirms the proper reading frame. (This is detailed in McDaniel, et al. 1988. J. Bacteriol. 170:2306-2311).

Armed with the DNA sequence and the known reading frame, a number of vector systems with different types of plasmids and different types of promoters are constructed and placed into E. coli. It is very difficult to improve on the expression that is observed in the normal soil bacteria source. This is evident if one compares the various bacterial strains and vectors in Table I. Extensive manipulation of these constructs demonstrates that the protein is deposited in the bacterial host membranes and that the E. coli membrane limits the amount of protein produced.

TABLE 1
Expression of opd in heterologous biological systems.

| Biological | Expression | | |
|---|---|---|---|
| host^{a} | Vector^{b} | Promoter^{c} | Activity |
| P. diminuta MG | pCMS1 (native) | opdP^{d} | 2.08 |
| (25° C.) | | | |
| P. diminuta MG | cured strain | none | <0.001 |
| (25° C.) | | | |
| E. coli JM103 | M13mp10 (phage) | lacP | 0.013 |
| (37° C.) | | | |
| E. coli JM103 | no phage | none | <0.001 |
| (37° C.) | | | |
| E. coli MC4100 | pLH540 (plasmid) | tacP | 0.020 |
| (37° C.) | | | |
| E. coli MC4100 | pLH540 (plasmid) | tacP | 1.10 |
| (25° C.) | | | |
| E. coli MC4100 | no plasmid | none | <0.01 |
| (25° C.) | | | |
| Sf9 cell culture | pLH1170^{e} | hedP | 12.50 |
| (25° C.) | | | |
| Sf9 cell culture | (uninfected)^{f} | none | <0.01 |
| (25° C.) | | | |

^{a}Temperature of growth conditions indicated in parentheses
^{b}Promoter utilized to express opd cistron
^{c}Enzymatic activity is express as 1 μmole of paraoxon converted to p-nitrophenol per milligram of protein per minute (units/mg) where E₄₀₀ = 17,000M⁻¹cm⁻¹
^{d}Native pseudomonad plasmid encoding opd
^{e}Baculoviral transfection of plasmid construction
^{f}Tested cells alone from uninfected and 360B-gal transfected cells

Thus, it is necessary to transfer the opd gene into an alternate host such as the baculoviral vector system. A recombinant DNA molecule is constructed using the polyhedron gene promoter to control synthesis of the opd transcript and this is transformed into insect tissue culture cells in the presence of a native helper virus. The transformed cultures produce high levels of OPA, up to 10-fold better than the original bacterial source (see Table I above).

Insect tissue cultures are screened for the presence of a clone demonstrating opd activity. Since paraoxon is degraded to form p-nitrophenol (yellow) plus diethyl thiophosphate, the relative rates of OP-hydrolysis by each clone is screened in microculture (250 microliters). Thus, cultured cell lines that are capable of producing 50 to 100 times the activity of the recombinant bacterial cultures may be selected.

### Use of Expression Vectors Comprising the opd Gene

The expression vectors comprising an opd gene fragment may be used to produce transgenic eukaryotic organisms. Such transgenic organisms are those that have had the genetic material of another organism inserted into their cells in some manner and have propogated the foreign DNA by incorporating it into their own cellular DNA. Typically, the foreign DNA is incorporated into the transgenic organism using a movable genetic element such as a transposon or a virus which is capable of infecting the transgenic host. For example, a transgenic fruit fly is produced by injecting an expression vector comprising a transposon carrying the opd gene into the fruit fly embryo cells. In another example, an expression vector comprising the baculovirus transfer vector carrying the opd gene is injected into Fall Army worm caterpillars. The transgenic insect is preferably a beneficial insect such as the honey bee or silk worm.

The recombinant OPA enzyme is produced using specific expression vectors. Each such vector must be comprised of a promoter and a start codon recognized by the host cell. The promoter may be selected from lac, tac, amp, the heat shock promoter of a P-element of Drosophila, or the baculovirus polyhedron gene promoter. In addition, the expression vector should include an opd DNA fragment in the correct orientation and reading frame with respect to the promoter sequence to allow translation of the opd gene. In one example, the expression vector comprises a plasmid such as pBR322. In another example, the expression vector comprises a bacteriophage such as bacteriophage M13.

In another example where transfer of the opd gene is to be effected in Drosophila, a plasmid carrying a transposon which in turn carries the opd gene sequence is required. Preferably, the Drosophila vector is a P-element with a heat shock promoter controlling opd. In the Fall Army worm, the expression vector is derived from the baculovirus enabling its use in transforming the insect cell lines. The baculovirus vector may be one of any of several baculovirus transfer vectors.

### Characterization of the Recombinant OPA and its Use

With the increased levels of production provided by the recombinant gene heterologously expressed in an insect host, purification of the enzyme in large amounts may be achieved. For example, from 8 g of insect cells infected with the baculovirus/opd vector, 2.7 mg of homogeneous enzyme may be obtained with an overall yield of 75% after 1500-fold purification.

The invention provides for purified enzyme with a specific activity of 3200 units/mg. Kinetic constants (k_{cat}, kₘ, k_{cat}/kₘ) can be calculated describing the catalytic efficiency of the enzyme. The kinetic values associated with the purified enzyme (specific activity=3200 units/mg) assuming a molecular weight of approximately 39,000 can be calculated as:

K_{cat}=2100 sec⁻¹

K_{cat}/Kₘ=4×10⁷ M⁻¹sec⁻¹

The pure enzyme is then used in a series of studies known to those skilled in the art to determine the mechanism of action and substrate specificity. (This is described more fully in Lewis, V. E., et al. 1988. Biochemistry 27: 1591-1597 and in Example III below).

This type of information allows one to predict what can and can not be accomplished with the enzyme relative to various types of substrates. For example, the purified enzyme may be used to determine whether this enzyme degrades mammalian neurotoxins such as Soman or VX.

An evaluation of the kinetic parameters for diisopropyl phosphonofluoridate (DFP) hydrolysis is also accomplished with the recombinant organophosphorus anhydrase using either a fluoride ion electrode or ¹⁹F-NMR. The Kₘ for DFP hydrolysis at pH 7.0 is 0.12±0.02 mM and the Vₘₐₓ is 3.6% of the maximal rate of hydrolysis of paraoxon. The Kₘ for paraoxon hydrolysis is 0.012±0.001 mM under the same reaction conditions for the enzyme produced in E. coli.

### Other Uses of the Invention

According to the method of this invention, detoxification using the purified OPA enzyme or the opd-containing microorganisms can be attained for a wide range of organophosphorus compounds. Detoxification is achieved by the initial hydrolysis across the susceptible bond of the organophosphorus compound. For example, detoxification of parathion may be achieved by conversion of parathion to p-nitrophenol and diethyl thiophosphoric acid. Detoxification using the purified OPA enzyme has the advantage of avoiding the potential release of genetically engineered microorganisms into the environment.

In one application of the invention, either the purified enzyme or the opd-containing recombinant microorganism is attached to a matrix allowing the construction of columns useful in detoxifying organophosphorous compounds. When a gas or fluid potentially contaminated with organophosphorus compounds is passed through the column matrix, the bound enzyme or microorganism reacts with the organophosphorus contaminant thereby detoxifying it. Likewise, in another application of the invention, the matrix-bound OPA or opd recombinant-microorganism is incorporated in a gas mask device to protect personnel such as pesticide applicators or soldiers exposed to hazardous levels of organophosphorus compounds present as vapors. In another application, a matrix-bound OPA or opd recombinant microorganism may be used to filter potentially contaminated air entering a closed environment such as a building or vehicle. In still another application, matrix-bound OPA or opd-containing recombinant microorganism contained within a solid phase column is used to detoxify an effluent. Such an effluent is a waste water stream from a locality where organophosphorus compounds are being manufactured, applied, or destroyed.

The invention may also be used to decontaminate a variety of loci by disseminating either the purified OPA or the opd-containing microorganism onto the locus. Any manner of dissemination may be used, but preferably the enzyme or the opd-containing recombinant bacteria will be sprayed preferably in an inert solution to better facilitate the spray. The potentially contaminated locus can range from a generally contaminated soil or body of water to military or commercial pesticide-application equipment. The locus can equally well be a pre- or post-harvest crop, an animal (including a human) or clothing. The locus may even be a stored foodstuff which has been treated or otherwise contaminated with an organophosphorus pesticide.

In another application of the invention, the purified enzyme or the recombinant opd microorganism may be a concentrated liquid form or a solid form such as a solid tablet. In such a concentrated formulation, the invention may be used to detoxify containers such as those used in commercial, agricultural, or domestic pesticide applications. Such a concentrated formulation may also be used in detoxifying containers of spent nerve gases. Concentrated formulations of the purified OPA enzyme or opd-containing recombinant bacteria may also be used as antidotes for poisons. These concentrated formulations may be used after poisoning or as a pretreatment for animals or humans prior to exposure to sub-acute or lethal doses of organophosphorus compounds.

In still another application of the invention, the recombinant microorganism comprising both the opd gene and an antibiotic resistance marker on the same vector is used in a plate assay to detect bacterial colonies capable of detoxifying organophosphorus compounds. Preferably, the control opd-microorganism will be mixed with samples of indigenous bacteria to provide an internal control to samples randomly obtained from soil, water, feeds, etc. By plating out aliquots of samples spiked with the control opd bacteria on both non-selective (no antibiotic) and selective (containing antibiotic) media, it is possible to calculate precisely the number of control opd-containing bacteria and to compare these with any indigenous bacteria potentially containing the opd gene. The presence of the control opd-containing bacteria provides a positive control enabling the method's effectiveness to be constantly monitored.

The plate assay may employ filters impregnated with an OPA substrate. Such a substrate is either chromagenic (as in the case of parathion, paraoxon, methyl parathion, etc.) or non-chromagenic relying on a differential absorption between the substrate and product (as in the case of coumaphos).

The plate assay filters are preferably selected for their ability to bind DNA or protein. After the bacterial colonies suspected of having the opd-gene are transferred to the surface of the filter, they are lysed and fixed to the filter's surface by methods known to those of skill in the art. Next, a radioactive probe specific for the opd-gene (DNA probe) or specific for the OPA enzyme (antibody probe) is used to hybridize to the filter. The DNA probe may be any portion of the DNA sequence of the opd gene fragment which is made radioactive by ³²P incorporation during the synthesis of the probe. Alternatively, the OPA enzyme is used to produce a polyclonal antisera which, if labelled radioactively with ¹²⁵I for example, can be used to probe for OPA in the bacterial samples.

The plate assay described above may also be used in an integrated pest management system. In this application of the invention, a pest management coordinator previews a soil or crop for the presence and quantity of bacteria capable of rapidly breaking down organophosphorus compounds using the plate assay results. With this information, the pest management coordinator selects the type, quantity and formulation of pesticides to apply to a soil or crop. For example, if the coordinator finds bacteria capable of breaking down organophosphorus compounds in the soil, the coordinator may likely select a formula or pesticide that comprises no organophosphorus compounds. Alternatively, the coordinator may coformulate organophosphorus compounds which compliment each other relative to being substrates and competitive inhibitors of the OPA enzyme.

As used herein, the term “co-formulate” (or “co-formulation”) refers to a combination of two or more organophosphorus pesticides where at least one pesticide is a preferred OPA substrate while at least one other pesticide is a competitive inhibitor of OPA.

In yet another application of the invention, the purified recombinant OPA enzyme may be used to detect the presence of trace amounts of organophosphorus compounds. This applications may be achieved in one of at least two ways. In one, OPA enzyme derived in a pure form from recombinant cells is used to pretreat aliquots of samples potentially containing organophosphorus compounds. Following this treatment, the sample and the OPA-treated aliquot are analyzed by standard pesticide residue analysis techniques. Detection of an organophosphorus compound in the untreated sample may be secondarily confirmed by selective removal or dilution by OPA in the treated sample.

In the other way, the purified recombinant OPA of the invention is used in a device wherein the enzyme is bound to a solid phase column matrix such as sepharose or DEAE cellulose and placed in a preferably small, portable column. A known volume of ambient air suspected of containing organophosphorus vapors is pumped through the column at a known rate. The exact volume of air and rate of flow can be determined based on the size of the column and the nature of the column matrix using commercially available columns and matrices. If the air contains an organophosphorus vapor sufficiently high in concentration to act as a competitive inhibitor of the enzyme, the matrix-bound OPA will be inactivated. By adding to the column a chromagenic substrate such as paraoxon, a measure of the amount of inactivation may be obtained. The measurement of the amount of substrate converted to product (i.e. color development associated with conversion of paraoxon to p-nitrophenol) may be achieved simply by visualizing the column eluate in comparison with a range of color standards. The color formation may also be monitored spectrophotometrically.

This application of the invention may be used, for example, to detect air potentially contaminated with a nerve gas. Air is passed onto the column, preferably comprising a solid phase column matrix, mechanically by using a syringe and 3-way valve. After a known volume of air is passed over the column the same syringe is used to load onto the column a volume of paraoxon-containing solution buffered at about pH 8.0. After a sufficient time (i.e. time enough for color formation to occur in an unexposed column) is allowed for reaction of the bound OPA with the paraoxon, the solution is driven from the column by forcing air into the column using the syringe. The color intensity generated by formation of p-nitrophenol from paraoxon is measured against a set of stable, color standards in order to ascertain the concentration of potential organophosphorus vapors in the ambient air. The more intense the color, the less nerve gas vapor there is in the air sample being tested. Alternatively, the comparison may include a blank column and a column treated with a known concentration of a competitive inhibitor of OPA. In this manner, a more exact estimation of the ambient concentration of organophosphorus vapors can be made. As discussed in Example IV below, at least two typically encountered nerve gases are competitive inhibitors of paraoxon hydrolysis by OPA. OPA may be covalently coupled to column matrices by methods known to those of skill in the art.

In another application of the invention, the recombinant OPA enzyme or the recombinant opd gene itself may be used to protect certain insects against organophosphorus poisoning. For example, insects such as silk worms localized to one vicinity may be dusted with purified recombinant OPA enzyme or with a recombinant microorganism comprising the opd gene. Alternatively, the recombinant OPA enzyme or opd-containing microorganism may be fed to a beneficial insect. Such feeding may be accomplished by adding the OPA enzyme or an opd-containing recombinant microorganism to the food supply such as leaves (in the case of silkworms) or to a food source of honey bees.

Beneficial insects may be protected by infecting such insects either topically or internally with opd-containing microorganisms. Most preferably, this “infection” is accomplished with microorganisms typically found in the natural flora of the insect's outer body or gut tract and transformed with a vector comprising opd. “Infection” of the insect with a naturally-associated microorganism carrying the opd-gene presents a greater likelihood that stable “infection” is achieved.

Most preferably, the opd gene itself is used to produce a transgenic insect which maintains the opd gene as a stable, inherited trait. At least two means for achieving such transgenic insects using the opd gene sequence of the invention are possible. In one means, the embryonic cells of an insect are microinjected with a vector carrying the opd gene in a transposon. The transposon used must be one which can insert itself into the host insect genome while carrying with it the opd gene. The construction of the vector is such that the opd gene is placed behind a heat shock promoter of a P-element naturally associated with Drosophila. In another means, the opd gene is incorporated into a vector which causes natural viral infection of the insect host. Such a vector carrying the opd gene of the invention is injected into larvae of the host insect. This means may be accomplished with the baculovirus transfer vector wherein the opd gene is placed behind the polyhedron promoter of that virus.

In still another application of the invention, purified, recombinant OPA enzyme may be used to predict successful co-formulations of organophosphorus pesticides to be used in field applications where said fields are suspected or known to contain bacterial populations capable of rapidly detoxifying organophosphorus pesticides. In this manner, the pesticide applicator selects a pesticide of choice for the given application. If the chosen pesticide is also identified by the invention as being a preferred substrate of the recombinant OPA, a co-formulation pesticide is selected from the group of organophosphorus pesticides which are competitive inhibitors of OPA relative to the preferred substrate. By allowing co-formulating in this manner, the invention provides for an extended half-life for the preferred substrate pesticide and extended control of the target pest.

### EXPERIMENTAL

The following examples illustrate various aspects of the invention. The examples should not be construed as limiting the claims.

### Example I

### Cloning and Sequencing of opd

Cloning and sequencing of the opd gene according to this invention may be accomplished, for example, as discussed using the bacterial strains and plasmids, media and growth conditions described below.

A. Bacterial Strains and Plasmids. P. diminuta MG is the original host of pCMS1. Escherichia coli strains HB101-4442 (auxotrophic for uracil and proline) and JM 103 were used as host cells for the cloning vectors, pBR322(12) and phage M13 mp10 (13), respectively. Hybrid gene fusions were produced in plasmid pMC1403 and expressed in E. coli CQ4(28).

B. Media and Growth Conditions. Cultures of bacteria were grown at 32° C. (P. diminuta) or 37° C. (E. coli). Nutrient medium consisted of 10 g of tryptone (Difco Laboratories), 5 g of yeast extract (Difco), and 5 g of NaCl per liter (TYE). TF minimal medium (14) was used for E. coli strains and was supplemented with uracil (50 μg/ml), proline (25 μg/ml), vitamin B₁ (0.01%), Casamino Acids (0.1%), glucose (0.2%), and antibiotics (25 to 50 μg/ml) as required.

C. Isolation of Plasmid DNA. Standard protocols for the isolation of DNA from E. coli for plasmid (15) or phage (13) have been described. Isolation of predominantly covalently closed circular plasmid DNA from P. diminuta was accomplished via a mild lysis procedure modified from that of Berns and Thomas (16).

D. Cloning and Sequencing of opd from the Native Plasmid. The PstI restriction fragments of pCMS1 were inserted into pBR322, inactivating the ampicillin gene (Focus 5:3, Bethesda Research Laboratories [BRL], Gaithersburg, Md., 1983). The resulting recombinant plasmids were used to transform competent HB101-4442, and tetracycline-resistant (Tc^{r}) colonies were selected and evaluated for ampicillin sensitivity (Ap^{s}). The plasmid structure of selected Tc^{r} Ap^{s} transformants were determined, and clones representing the different inserts were analyzed for activity.

The 1.3-kb PstI insert of pBR322-038 was excised from its vector, purified by preparative agarose gel electrophoresis using a modified freeze-squeeze phenol procedure (S. A. Benson, Biotechniques March/April: 66-67, 1984), and subsequently introduced into the multiple cloning site of M13 mp10. The resulting recombinant molecules were transformed into competent E. coli JM103 cells, and clear-plaque isolates were selected. All subsequent manipulations of viral recombinant DNAs were performed according to the methodology of the Bethesda Research Laboratories (BRL) “M13 Cloning/Dideoxy Sequencing Manual.” A variety of 5′ and 3′ deletions of pBR322-038 were constructed, using various restriction sites surrounding the opd gene (BamHI, AvaI, NruI, SalI, SphI). In addition, 3′ exonuclease III deletions were used to identify gene boundaries.

Dideoxy sequencing was accomplished by the method of Sanger as detailed in the BRL “M13 Cloning/Dideoxy Sequencing Manual.” In cases where GC compaction was evident, reverse transcriptase as well as the Klenow fragment of DNA polymerase was used (BRL, manufacturer's protocols). Oligonucleotide primers were synthesized using phosphoramidite chemistry with an Applied Biosystems Synthesizer according to the manufacturer's recommendations.

The 5′ region of the opd gene was subcloned into the β-galactosidase gene for the purposes of producing a lacZ fusion, polypeptide. The 1.3-kb opd fragment was restricted with AvaI; the staggered restriction fragment was end-filled and ligated into the 5′ SmaI cloning site of the lacZ fragment of pMC1403(28). This hybrid genetic construction was then transformed into E. coli CQ4(17).

E. OPA Enzyme Assay. Routine analysis of parathion hydrolysis in whole cells was accomplished by suspending cultures in 10 mM Tris hydrochloride (pH 8.0) containing 1.0 mM sodium EDTA (TE buffer). Cell-free extracts were assayed using sonicated extracts in 0.5 ml of TE buffer. The suspended cells or cell extracts were incubated with 10 μl of substrate (100 μg of parathion in 10% methanol), and p-nitrophenol production was monitored at a wavelength of 400 nm. To induce the gene under lac control, 1.0 μmol of isopropyl-β-D-thiogalactopyranoside (Sigma) per ml was added to the culture media.

F. Column Chromatography, Affinity Chromatography, and Protein Sequencing. P. diminuta cells from a 200-liter fermentation were harvested by a continuous-flow centrifuge and suspended in 2.0 liters of 1.0 M NaCl. Samples of this suspension were agitated in a Waring blender for 30 s, and the resulting suspension (5.0 ml) were sonicated, treated with 0.1% Triton X-100, and stirred at room temperature for 2 h before chromatography.

The molecular weight of the native enzyme was determined by ascending Sephadex G-200 chromatography in the presence of 50 mM CHES buffer [2-(N-cyclohexyl-amino)ethanesulfonic acid (pH 9.0)] at 4° C. Enzymatic activity was located by introducing 50-μl aliquots of column fractions (2.0 ml) into a reaction volume of 0.8 ml containing 0.2 mM paraoxon and 50 mM CHES buffer (pH 9.0).

Purification of hybrid β-galactosidase proteins encoding the 5′ region of the opd gene was achieved by immunoaffinity chromatography and preparative gel electrophoresis. Gas-phase sequencing of the purified fusion polypeptide (Applied Biosystems 470A Sequencer, Applied Biosystems 120A On-Line-PTH Analyzer, was accomplished by the methods of Hewick, et al. (18).

G. Cloning of pCMS1 into PBR322. The entire DNA from the degradative plasmid was digested with PstI (generating fragments of approximately 18.5, 17.3, 5.3, 4.3, 1.7, 1.6, 1.3, and 0.8 kb) and was subcloned into pBR322 within that vector's ampicillin gene. Cell-free lysates of Ap^{s} clones selected from the Tc^{r} transformants of E. coli HB101-4442 were tested for activity. A single-colony isolate was selected for its ability to hydrolyze parathion, and the expected phenotype (Tc^{r} Ap^{s}; auxotrophy for uracil and proline; parathion hydrolysis) was verified. A 5.6 kb, CsCl-purified plasmid isolated from this strain was used to transform competent HB 101-4442 cells, regenerating the phenotype and demonstrating that the hydrolytic activity is mediated by the recombinant plasmid.

Insertion of the 1.3-kb PstI fragment into the multiple cloning site of M13 mp10 produced an opd-encoding phage (M13 mp10-038/008) possessing an inducible (isopropyl-β-D-thiogalactopyranoside) whole-cell activity in E. coli JM103. This phage may be used in hybridization studies (“C-tests”) to select other isolates which possess similarly sized insertions but lacked activity.

H. Nucleotide Sequencing. Dideoxy sequencing along both strands of the opd gene revealed a potential translational reading frame (FIG. 1). Oligonucleotide primers were constructed for the purposes of sequencing regions lacking convenient restriction sites. In all cases, these primers were selected to efficiently promote DNA synthesis.

The open reading frame begins 12 base pairs from the 5′ PstI site. A potential start site (ATG) was located 17 codons into the open reading frame. This codon appeared to be a candidate for the translational start since it is preceded by an AAGCAA sequence 15 base pairs upstream; the sequence and spacing are in good agreement with known Pseudomonas ribosomal binding sites (19). An N-terminal deletion of some of the sequence prior to the ATG start codon is possible without complete loss of activity. In addition, several potential Rho-dependent terminator structures ranging in free energy of association from −12.6 to −15.4 kcal/mol (ca. −52.7 to −64.4 kJ/mol) were located 3′ of the open reading frame.

I. Amino Acid Sequencing of Fusion Polypeptides. When a fusion protein is constructed between the 5′ region of the opd gene and the lacZ gene at the AvaI-SmaI site, a hybrid polypeptide can be recovered, purified, and subjected to amino acid sequencing. This confirmed the reading frame.

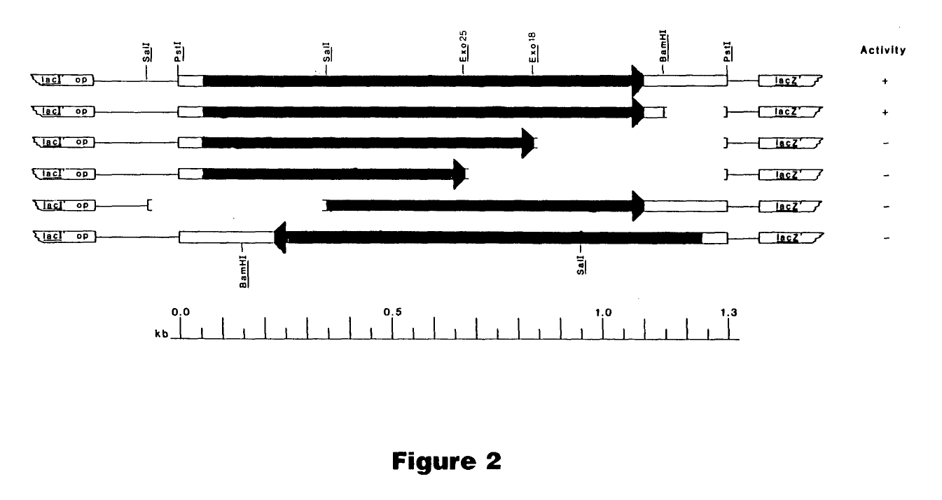

J. Subcloning regional deletions. FIG. 2 summarizes results obtained with various subclones of the 1.3-kb fragment containing the opd gene. Deletions outside the putative coding region remain active when the sequence is properly oriented for expression from the lacZ promoter. If the orientation is reversed or if deletions are made within the putative coding region, activity is eliminated. In particular, it was possible to remove the C-terminal BamHI-PstI fragment without significant loss of activity.

### Example II

### Heterologous Expression in Insect Tissue Culture

Heterologous expression of opd in insect tissue culture may be obtained according to this invention as described in the example below.

A. Molecular Manipulations of Recombinant DNA Vectors. Standard recombinant DNA techniques were employed (20), using enzymes purchased from either Bethesda Research Laboratories, Gaithersburg, Md., or Boehringer Mannheim Biochemicals, Indianapolis, Ind. A BamHI restriction fragment, containing the entire opd open reading frame, 62 bp of the 5′ flanking sequence, and 17 bp derived from the polyclonal region of M13 mp10, is isolated from the RF DNA of clone mp10-008. This 1170 bp fragment was subcloned into the BamHI site of the tac promoter vector pDR540, obtained from Pharmacia, Inc., Piscataway, N.J., and the resulting construction was designated pLH540opd was transformed into the lacI-E. coli strain MC4100 (17). This same BamHI fragment was subsequently isolated from pLH540opd and cloned into the BamHI site of the baculovirus transfer vector pVL941 (21). The resulting construction was designated pLH1170opd and was a derivative of pAC311 in which the polyhedron ATG start has been mutated by site-directed mutagenesis to an ATT triplet. Translation of a foreign protein expressed from the polyhedron promoter in pLH1170 pd is thus initiated at the first ATG codon in the foreign gene's ORF, and a non-fused protein is produced. Expression of the native opd sequence is under the control of the baculoviral polyhedron promoter (hed-promoter).

B. Production of recombinant virus. Spodoptera frugiperda (fall armyworm) sf9 cells (22) were co-transfected with wild-type (AcMNPV) viral DNA and pVL941-29 by a modification of the calcium phosphate precipitation technique. The transfected cultures were incubated for 5 days in TMN-FH media (23) supplemented with 10% (v/v) fetal calf serum at 27°, after which the supernatants were removed from each well and saved as viral stocks. The cells in each well were lysed by the addition of 200 ul of 0.5N sodium hydroxide. The lysates were neutralized by the addition of 20 ul of 10 M ammonium acetate to each sample, and each lysate was then spotted onto a nitrocellulose filter. The filter was baked for 2 hrs at 800, and the lysates were screened by hybridization with a nick-translated, ³²P-labelled, opd-specific probe. Clones were selected for further purification and analysis on the basis of their strong hybridization signal. A second transfection was performed as above, and recombinant plaques were transferred to a 96-well plate. After 2 days at 27° C., the infected wells were assayed directly for expression of the opd gene product by the addition of ^{˜}100 ul of 3.6 mM paraoxon (pH 7) to each well. The plate was incubated for 2 hrs at 27° C. opd+ recombinants were detected by the enzymatic release of p-nitrophenol, producing an intense yellow color in opd+ wells. Isolates were selected for further purification and analysis.

C. Relative enzymatic activity of bacterial and baculoviral opd constructions. Table I summarizes an examination of the relative expression of the opd by measuring the activity of its enzymatic product. The hydrolysis of paraoxon under standard conditions was used for comparative purposes and the enzymatic activity is expressed as 1 μmole of paraoxon hydrolyzed to diethylphosphate and p-nitrophenol per milligram per minute (units/mg protein). Activity was measured at 25° C. in a Gilford Response Spectrophotometer or Gilford model 260 spectrophotometer (ξ₄₀₀=17,000 M⁻¹ cm¹). The levels of expression in E. coli strains with various promoters (lac, tac, bla and other constructions at 37° C. were disappointing and never exceeded 3-4% of that obtained in the native Pseudomonas diminuta MG source). This difference is even more dramatic than apparent since the expression from the lacP was attempted on high copy plasmids (pBR322-based constructions) and M13-constructions (best expression reported in Table 1). The use of the tacP of the expression vector pDR540 does not produce increased expression under conditions of IPTG-induced expression in the MC4100 E. coli host background.

When expression studies are shifted from bacterial systems to baculoviral transfection in insect tissue culture, further production can be realized. In Sf9 cell cultures under expression of the hedP promoter of polyhedron from Autographa californica, a nuclear polyhedrosis virus, the enzymatic activity is increased to 10-15 units per mg protein in the primary culture. This permits the complete purification of the enzyme.

Expression requires transfection with the wild-type virus for complete infection and development.

### Example III

### Purification of OPA & Substrate Specificity

Purification of the recombinant OPA from insect tissue culture may be achieved according to this invention as described in the example below.

A. General—Enzymatic activity was measured by monitoring the absorbance at 400 nm as 0.75 mM paraoxon was hydrolyzed to diethylphosphate and p-nitrophenol (ε₄₀₀=17,000 M⁻¹ cm⁻¹) in 150 nM CHES, pH 9.0, buffer using a Gilford model 260 spectrophotometer regulated at 25&° C. One unit of activity was defined as the hydrolysis of 1 μmole of paraoxon/min. Protein concentration in crude samples was determined by measuring the absorbance at 280 nm or by the bicinchoninic acid assay method developed by Smith et al. (24) (Pierce Chemical Co.) with bovine serum albumin as a standard. Denaturing polyacrylamine gel electrophoresis was carried out by the method of Laemmli (25) and protein was visualized by silver staining according to the method of Wray, et al. (26).

B. Purification of the enzyme—The enzyme from Pseudomonas diminuta was purified from sf9 cells (fall armyworm) infected with the recombinant baculovirus (pVL941-29) containing the opd gene. The cells were infected at a cell density of 2−2.5×10⁶ cells/mL with 0.2 mL virus/mL cells. The virus generally should have a titer of 1×10⁸ (plaque forming units)/mL. This infection was allowed to proceed at 27° C. for four days before harvesting the cells by centrifugation at 6100×g for 30 min at 4° C. All subsequent steps in the purification were carried out at 4° C. The baculovirus infected sf9 cells (5-6 g/L of cell culture) were resuspended in 50 mM triethanolamine pH 9.0 buffer Cs containing 0.1 mM lnCl₂ (buffer A) and gently stirred for one hour. Cell lysis was achieved by 5 sec pulsed-sonication for 5 min at a medium power setting using a Heat Systems—Ultrasonics, Inc. model W830 ultrasonic processor with a macro-probe tip. This suspension was centrifuged at 25,000×g for 30 min. The supernatant fluid was decanted and the cells were resuspended in buffer A and centrifuged as before. This supernatant fluid is combined with the previous supernatant fraction. DEAE-cellulose (DE-52, Whatman), washed and equilibrated in buffer A, was added to the combined supernatant fractions at a concentration of 1 mL settled gel per 500 mg protein. This slurry was swirled for 30 min and filtered through a coarse scintered glass funnel, retaining the filtered solution for application to a 2.5×48 cm Green A dye matrix column (Amicon Corp.) equilibrated in buffer A. The enzyme was applied at a rate of 1 mL/min. The column was extensively washed with buffer A before initiating a 800 mL, 0-700 mM KCl gradient in buffer A at a rate of 1.0 mL/min. The fractions containing enzyme activity are pooled and loaded onto a phenyl sepharose column (2.5×15 cm) equilibrated in buffer A containing 700 mM KCl. After loading at a rate of 1 mL/min, the column was thoroughly washed with buffer A. The enzyme was eluted in a 800 mL 0-60% ethylene glycol gradient in buffer A at 1.0 mL/min. The fractions containing the enzyme were pooled and aliquots of 30 mL were loaded on a 2.5×90 cm G-75 Sephadex column equilibrated in buffer A and chromatographed.

C. Purification—Table 2 summarizes the results of a typical purification procedure. Elution of the enzyme with 700 mM KCl resulted in a 20-fold purification. Moreover, the relatively high salt concentration aided in the hydrophobic interaction between the enzyme and the phenyl sepharose media used in the subsequent column. The introduction of ethylene glycol into the phenyl sepharose column allowed elution of the enzyme without the denaturing side effects often observed with other organic solvents. This purification step provided an additional 16-fold purification. The enzyme could be further purified by gel filtration to give a homogeneous preparation. From approximately 8 g of pVL941-29 infected sf9 cells, approximately 2.7 mg of homogeneous enzyme were obtained with an overall yield of 75% after a 1500-fold purification.

TABLE 2
Purification of Phosphotriesterase

| | | | | Specific | | |
|---|---|---|---|---|---|---|
| | Volume | Activity | | Activity | Fold | |
| Step | (mL) | (mmol/min) (mg) | Protein | (units/mg) | (%) | Yield |
| Sonicate | 226 | 11400 | 5400 | 2.1 | 1.0 | 100 |
| DE-52 | 450 | 11800 | 1550 | 7.6 | 3.6 | 104 |
| Green A | 124 | 9390 | 62.4 | 150 | 71 | 82 |
| Phenyl | 117 | 10100 | 4.2 | 2400 | 1140 | 89 |
| Sepharose | | | | | | |
| G-75 | 390 | 8520 | 2.7 | 3200 | 1500 | 75 |

D. Substrate Specificity—Organophosphorus compounds were evaluated as substrates at pH 9 in 100 mM CHES buffer. The reactions were monitored spectrophotometrically, either in the UV region or within the visible spectrum depending upon the characteristics of the substrate. Pesticides such as O,O-diethyl-O-[3-chloro-4-methyl-2-oxo-2H-lbenzopyran-7-yl]phosphorothioate (coumaphos), O,O-dimethyl-p-cyanophenyl phosphorothioate (cyanophos), O,O-diethyl-O-[2-isopropyl-4-methyl-6-pyrimidyl] phosphorothioate (diazinon), O,O-diethyl O-[3,5,6-trichloro-2-pyridyl] phosphorothioate (dursban), O,O-diethyl [p-(methylsulfinyl) phenyl] phosphorothioate (fensulfothion), O,O-diethyl-O-p-nitrophenyl phosphorothioate (parathion), and O,O-dimethyl-O-p-nitrophenyl phosphorothioate (methyl parathion) were purchased from Chem Service, Inc., West Chester, Pa. The limited solubility of these compounds necessitated the addition of 10% methanol to the reaction mixtures.

E. Substrate Specificity—There was an extensive set of commercially used organophosphate pesticides that are hydrolyzed by the recombinant OPA enzyme (Table 3). The OPA enzyme from Pseudomonas diminuta will hydrolyze many of the commonly used organophosphorus insecticides in addition to paraoxon. Replacement of the phosphoryl oxygen with a sulfur increases the Kₘ but reduces Vₘₐₓ. Substitution of methoxy for ethoxy groups produces substrates with higher Km values and reduced catalytic rates. The size of the leaving group appears to be relatively unimportant since coumaphos was hydrolyzed at a rate comparable with parathion. This suggests that there are probably few molecular interactions between the enzyme and the leaving group. The dominant factor in the rate of substrate hydrolysis was stabilization of the anionic product (This aspect is further described in Lewis, et al. 1988. Biochemistry 27: 1591-1597).

The experimentally determined value for k_{cat} and k_{cat}/Kₘ with paraoxon as a substrate are substantial. The k_{cat}/Kₘ of 4×10⁷ M⁻¹ s⁻¹ was very close to the diffusion controlled limit of 10⁸-10⁹ M⁻¹ s⁻¹. Thus, the enzyme activity of this protein was quite respectable.

TABLE 3
Kinetic Constants for the Hydrolysis of
Organophosphorus Insecticides

| Structure | Common Name | wavelength (nm) | Kₘ (mM) | V_{ref} | (V/K)_{ref} |
|---|---|---|---|---|---|
| 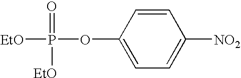 | paraoxon | 400 | 0.09 | 100 | 100 |
| 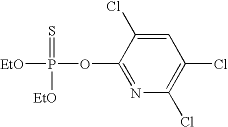 | dursban | 276 | 0.11 | 0.08 | 0.07 |
| 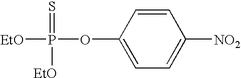 | parathion | 400 | 0.24 | 30 | 11.25 |
| 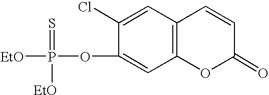 | coumaphos | 348 | 0.39 | 29 | 6.70 |
| 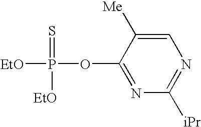 | diazinon | 228 | 0.45 | 8.4 | 1.68 |
| 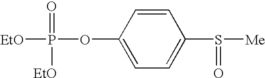 | fensulfothion | 284 | 0.46 | 3.2 | 0.63 |
| 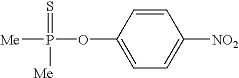 | methyl-pararthion | 400 | 0.84 | 2.4 | 0.26 |
| 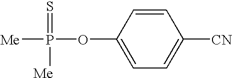 | cyanophos | 274 | 2.1 | 7.5 | 0.32 |

F. Amino Acid Composition—The amino acid composition was determined for a homogeneous preparation of OPA enzyme and compared favorably with the predicted composition from the DNA sequence of the opd gene. The N-terminus could not be sequenced, apparently due to some terminal modification of the protein.

### Example IV

### Detoxification of Nerve Agents

The organophosphorus-degrading genes (opd) isolated from plasmids of Pseudomonas diminuta and Flavobacterium encode identical Organophosphorus Acid Anhydrases (EC 3.1.3.-) which are capable of hydrolyzing a wide spectrum of insect and mammalian neurotoxins. The Pseudomonas enzyme can be purified following expression from a recombinant baculoviral vector in insect tissue culture of the Fall Armyworm, Spodoptera frugiperda (Sf9 cells). Purified enzyme preparations have been shown to be able to detoxify a number of structurally related acetylcholinesterase inhibitors including the organophosphorofluoridate nerve agents, sarin and soman. This was the first recombinant DNA construction capable of degrading these potent nerve gases. This enzyme was capable of degrading the common organophosphorus insecticide, paraoxon, at rates exceeding 2×10⁷ M⁻¹ (mole enzyme)⁻¹ which are equivalent to the most catalytically efficient enzymes observed in nature. The purified enzyme preparations are capable of detoxifying O,O-diisopropyl phosphorofluoridate (DFP), a less toxic model mammalian neurotoxin, and 1,2-dimethylpropylmethyl-phosphorofluoridate (sarin) at equivalent rates (50-60 molecules per molecule of enzyme per second). In addition, the enzyme can hydrolyze 1, 2, 2 trimethylpropylmethyl-phosphorofluoridate (soman) at approximately 10% of the rate of Sarin. The breadth of substrate utility and the efficiency for the hydrolysis exceeds the known abilities of other prokaryotic and eukaryotic organophosphorus acid anhydrases and it is clear that this detoxification profile was due to a single enzyme rather than a family of related, substrate-limited proteins.

### Example V

### Protection of Insects with OPA

Insects may be protected according to this invention using recombinant opd-containing microorganisms or purified OPA in a manner similar to the example below. The example describes use of crude enzyme extract only.

A. Wild type Drosophila melanogaster propagated by standard methods (27) by using a medium containing per liter: 100 g glucose, 10 g agar, 100 g yeast and 3 g p-hydroxybenzoate as a fungistat. Adult flies of approximately the same age were used for each assay to avoid any fluctuation in pesticide susceptibility due to age differences. Flies were anesthetized with ether before being transferred and checked for their full recovery before exposure to the pesticide in testing vials.

B. Flavobacterium sp. (ATCC #27551) were grown in nutrient broth containing per liter: 10 g tryptone, 5 g yeast extract, and 10 g sodium chloride. Overnight cultures were harvested by centrifugation, and the cell pellet was washed and dried three times with acetone. The hard pellet was then pulverized with mortar and pestle. The resulting dried cell powder can be and was stored at 4° C. for several months. For the bioassay, a fresh OPA enzyme solution was made by dissolving 0.05 g of dry cell powder in 5 ml of Tris-HCL buffer (10 mM, pH-8.5).

C. Small double filter papers (Whatman 984H, 2.4 cm size) were impregnated with exact amounts of pesticide solution by means of a microsyringe. The solvent (2, 2, 4-trimethylpentane, hexane, acetone, or ethyl acetate) was allowed to evaporate for 30 min. Control filters impregnated with the same volume of solvent showed no acute affect and indicated that 30 min. was sufficient to allow evaporation of all of the solvent.

D. A fixed amount of either PTE enzyme solution or buffer was also impregnated on the filters and incubated at 37° C. for 30 min. to allow pesticide degradation. At the end of the incubation, exactly 10 flies along with a few drops of sugar or honey water were added into each testing vial containing the filters. The vials were allowed to stand at room temperature overnight (between 16 to 20 h) and the percent fly survival in each vial was determined by visual inspection.

E. Parathion was one of the more commonly used pesticides and was also a good substrate for the OPA enzyme. Results in Table 4 show that all the flies were killed by parathion in the test vials where PTE was not added. Even at the lowest amount of parathion applied (1.2 μg) no fly survived overnight. In contrast, all the test flies survived where the pesticide-impregnated filters were treated with OPA (0.87 mg) prior to the fly test.

TABLE 4
Crude Enzyme Effect on Certain Organophosphorus Pesticides.

| | | Crude | Percent |
|---|---|---|---|
| Pesticide | (μg) | enzyme (mg) | survival |
| Parathion | 1.2 | 0 | 0 |
| | 1.2 | 0.87 | 100 |
| | 12.4 | 0 | 0 |
| | 12.4 | 0.87 | 100 |
| | 41.1 | 0 | 0 |
| | 41.1 | 0.87 | 100 |
| | 82.6 | 0 | 0 |
| | 82.6 | 0.87 | 50 |
| | 82.6 | 1.74 | 100 |
| | 124 | 0.87 | 0 |
| Diazinon | 11.0 | 0 | 0 |
| | 11.0 | 0.87 | 100 |
| | 22.0 | 0 | 0 |
| | 22.0 | 0.87 | 100 |

When the amount of parathion was increased to 82.6 μg, only 50% of the flies survived in OPA-treated test vials, and suggested that more enzyme may be needed for detoxifying all the parathion. This was found to be true when the amount of OPA was doubled, as also shown in Table 4. These results indicated that to get a conclusive result, it was important not to have an excessive amount of pesticide so that the amount of OPA becomes the limiting factor. This can be achieved by performing multiple tests using different dilutions of the same sample. Essentially the same results were obtained with diazinon (Table 4).

F. Since certain organophosphorus pesticides, and other insecticides such as carbamates are not degraded by the OPA enzyme, one would predict that the survival in the bioassay should not be affected by the enzyme treatment. Results shown in Table 5 for certain organophosphorus and carbamates pesticides, respectively, indicate that for a wide range of pesticide concentrations, there was no difference in survival between enzyme-treated and untreated samples. This demonstrates the basis of this modified fly test which was to rely on the specificity of OPA toward detoxification of certain pesticides.

TABLE 5
Crude Enzyme Effect on Certain Organophosphorus
Pesticides and Carbamate Pesticides

| | | Crude | Percent |
|---|---|---|---|
| Pesticide | (μg) | enzyme (mg) | survival |
| Fenthion | 1.5 | 0 | 0 |
| | 1.5 | 0.87 | 0 |
| | 7.7 | 0 | 0 |
| | 7.7 | 0.87 | 0 |
| | 46.0 | 0 | 0 |
| | 46.0 | 0.87 | 0 |
| Carbofuran | 1.0 | 0 | 100 |
| | 1.0 | 0.87 | 100 |
| | 7.6 | 0 | 20 |
| | 7.6 | 0.87 | 10 |
| | 11.5 | 0 | 0 |
| | 11.5 | 0.87 | 0 |
| | 22.9 | 0 | 0 |
| | 22.9 | 0.87 | 0 |
| Aldicarb | 9.5 | 0 | 0 |
| | 9.5 | 0.87 | 0 |

### Example VI

### Protection of Insects with Recombinant opd Gene

A recombinant baculovirus containing the opd gene was constructed using recombinant DNA techniques as described previously. The open reading frame from vector pDR540-1 was inserted into the baculovirus vector pVL941 (21).

To assess whether the expression of a functional OPA enzyme within living insects will protect against the lethal effects of an insecticide, the recombinant virus was injected directly into the larval stage of the fall army worms. The caterpillars were injected late in the third instar by taking up 5 μL of 2×10⁸/mL pVL941-29 into a fine capillary needle and injecting it into the hemolyph through one of the prolegs. After a relatively short lag phase, activity was detected in the caterpillar when paraoxon was used as a substrate. For the first four days after injection, the enzyme activity increased about an order of magnitude every 24 hours until it reached a maximum of approximately 11 units of paraoxon hydrolyzing activity per caterpillar. No enzyme activity (<5×10-⁵ units/caterpillar) was detected in uninfected caterpillars or caterpillars infected with the wild-type baculovirus.

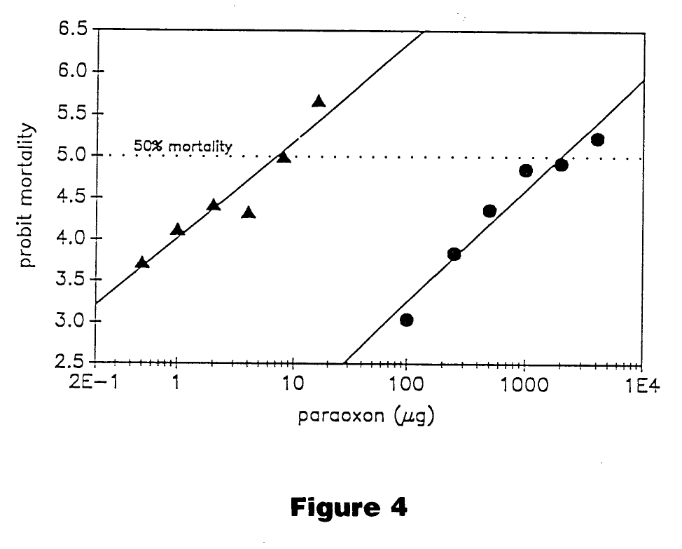

The effect of the insecticide paraoxon can be determined on both the infected and uninfected caterpillars. Shown in FIG. 4 is a plot of mortality versus the amount of paraoxon that can be applied directly to the caterpillars. This graph demonstrates that those caterpillars containing a functional OPA are resistant to all but the highest concentration of paraoxon that was applied. The LD₅₀ for the pV941-29 infected caterpillars was calculated to be 2000±XX μ while the control group (containing a mixture of uninfected and pAC311 infected catepillars) had an LD₅₀ of 9±XX μg. Thus, the lethal dose increased by a factor of at least 220 due to the presence of the opd gene. In the moth stage the LD50 was found to be 1 μg of paraoxon.

The results presented above demonstrated that resistance to paraoxon and other pesticides can be induced in insects by expression of an enzyme that was known to efficiently hydrolyze these molecules to nontoxic products. It should, therefore, be feasible to construct alternative systems involving the incorporation of the recombinant opd gene into other insect species.

### Example VII

### Chromagenic Assay Using opd+ Controls

A. Bacterial Strains and Media Pseudomonas diminuta MG was the original host of pCMS1. Cultures were grown at 32° C. on nutrient media consisting of 10 g Bacto-tryptone, 10 g Bacto-yeast extract, and 5 g of NaCl per liter (TYE). Ps. diminuta strains were maintained as 40% glycerol stocks at −70° C.

B. Plate Assay Technique Filter pads (8.5 cm) of Whatman No. 1 filter paper were treated by spraying evenly with a 2-3 ml volume of a 10 mg/ml solution of technical parathion (Monsanto) in methanol. The pads were forced-air dried and stored in the dark at room temperature until used.

Pesticide-impregnated pads were applied to the surface of TYE plates grown at 32° C. and containing colonies which were allowed to grow to a diameter of 2-4 mm. The colony populations of each plate were lifted off the surface onto the treated pad. Parathion hydrolysis was permitted to continue for 30 min at 37° C. in a humidified incubator at pH 9.0. Single colonies of non-degrading strains (opd−) were identified from among parathion-degrading strains (opd+) on plates by the appearance of the yellow product, 4-nitrophenol. Conversely, rare opd+ isolates were selected from among numerous opd− colonies. In all cases, the pads were marked appropriately to allow for further reference and the master plate was reincubated for regeneration of the original colonies.

C. UV-Photography and Eclipsing Method The described method relies on the release of a chromogenic product (4-nitrophenol) and the absorption of ultraviolet irradiation (maximum absorbance=400 nm, molar extinction coefficient=1.88×10⁻⁴). UV-enhanced photography of filter pads was accomplished by first photographing (tungsten filament) the master plate prior to lifting off the colonies onto the treated pad. A treated pad was used to lift the master colonies off the plate and after sufficient time has been allowed for 4-nitrophenol development, a photograph is taken, using a combination of short (254 nm) and longwave (366 nm) ultraviolet irradiation.

The negatives of the two exposures are aligned in such a way as to precisely overlay the colonies of the lift negative above those same colonies on the master plate; the dark 4-nitrophenol producing (UV-absorbing) colonies of the pad lift eclipsed the bright, white colonies of the original master plate.

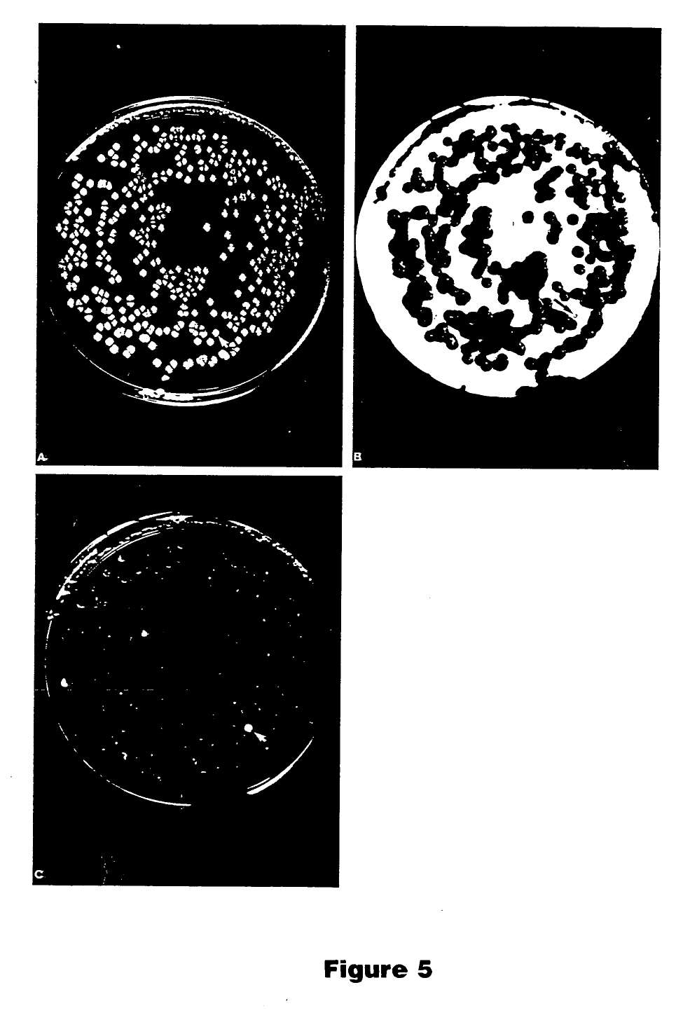

D. Results. FIG. 5a shows a TYE plate containing approximately 500 colonies of Pseudomonas diminuta photographed as described. It was possible to identify opd− colonies sufficiently separated from surrounding opd+colonies. Such a colony was indicated in FIG. 5a.

However, in order to quantitate large numbers of closely packed colonies, the UV-absorbing characteristic of 4-nitrophenol were used to produce a black and white image of the developed (4-nitro-phenol coloration) filter lift. FIG. 5b shows the results of such a photograph of the filter lift produced when the master plate in FIG. 5a was blotted with a parathion-impregnated filter pad. The colony previously identified by visualizing 4-nitrophenol development under white light is seen as a non-absorbing spot in contrast with the majority of opd+ colonies which appear black using this photographic method.

FIG. 5c demonstrates the photographic enhancement which permits the identification of rare colonies among colonial masses which no longer possess the phosphotriesterase activity. This image was produced as described by eclipsing the two negatives of the photographs in FIGS. 5a and 5b. The resulting image indicated at least two additional opd− colonies seen here as half moon-shapes in addition to the one originally identified in FIGS. 5a and b. Using this technique, it was possible to quantitate and isolate opd− derivatives from the parental opd+Pseudomonas diminuta strain.

As a test of the sensitivity of the method for identification of rare opd+ colonies among many opd− colonies, opd+ cells of Pseudomonas diminuta MG were mixed with cells of an opd− derivative of that strain. Ratios of positive to negative phenotypes vary from 1/100 to 1/100,000. At all dilutions, the rare opd+ colonies were visible. Plates containing as many as 10,000 colonies were readily screened with a 95% efficiency.

### Example VIII

### Detection of opd-Containing Bacteria Using DNA Probes

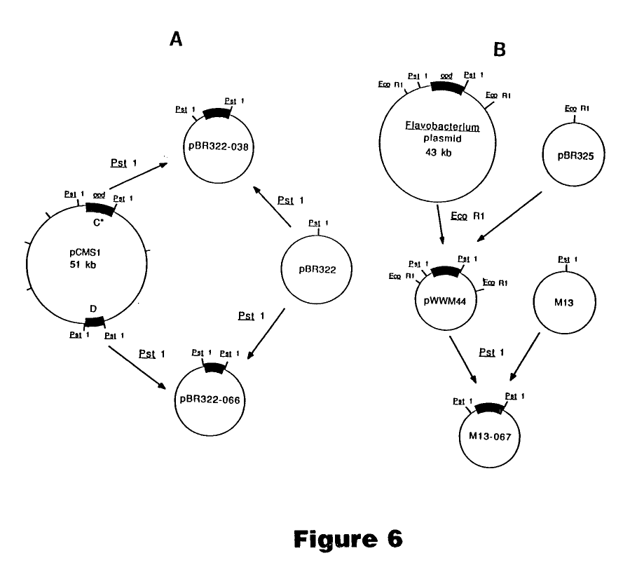

A. Subcloning Test Fragments. The nonidentical nature of the two separately isolated plasmids was demonstrated by using a pair of PstI fragments from the P. diminuta plasmid (pCMS1) as probes against the plasmid DNA of the Flavobacterium sp. (FIG. 6). The cloning of the two PstI fragments (C″ and D) from pCMS1 has been described previously (Example I). The C″ fragment from the Pseudomonas plasmid (1,326 bp) containing the opd gene was shown by sequence analysis to be identical to the same-size fragment from the Flavobacterium plasmid. A second PstI fragment (D) of approximately 900 bp was chosen as a probe since it was separated from the region containing the known homology by approximately 22 kb, as estimated by a preliminary restriction digest map of the Pseudomonas plasmid. For all of the hybridization studies, the methods of Southern were used (29).

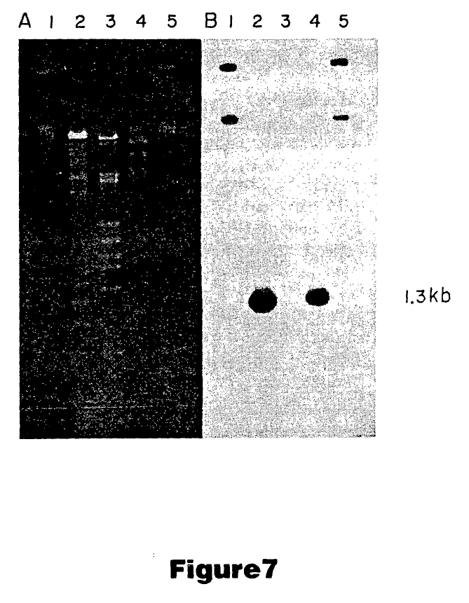

B. Identity of opd Fragments. FIG. 7 demonstrates the strong hybridization of both Pseudomonas and Flavobacterium plasmid DNAs with the 1,325-bp (C″) fragment containing the opd gene sequence. The PstI-digested plasmids differed considerably in their restriction profiles (FIG. 7A). There appears to be a single plasmid in the Flavobacterium strain, although it was present in several forms. Upon restriction, a single hybridizing band was observed for each of the two plasmid sources of the gene (FIG. 7B), and the overall restriction endonuclease pattern was similar to that observed for the isolated plasmid.

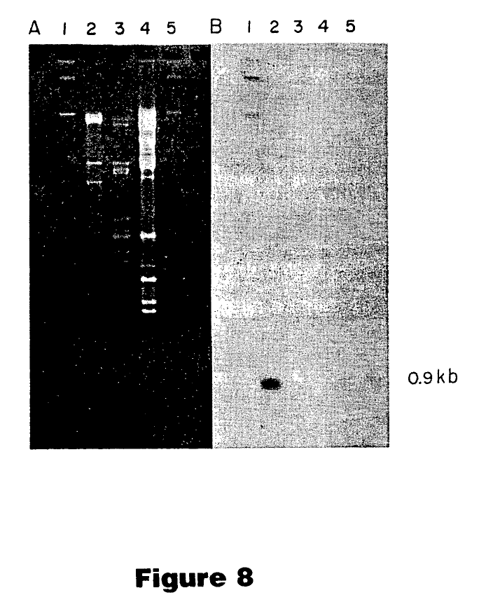

When the 900-bp fragment (D) was used as a probe against both plasmid DNAs (FIGS. 8A and B), it hybridized to DNA in the control (PstI-digested pCMS1) and the unrestricted Pseudomonas plasmid. However, the 900 bp fragment failed to hybridize to either the native or the restricted plasmid DNA from Flavobacterium sp. These results were consistent with the restriction site data and reiterated the dissimilarity of the two plasmids.

C. Screening Other Strains for opd using DNA Probes. Table 3 summarizes studies in which 8 different bacterial systems were evaluated for sequence similarity to the opd gene sequence in order to evaluate whether those bacterial systems carry hybridizing DNA sequences and compare paraoxonase activity with DFPase activity. It was possible with the present invention to screen microorganisms in order to find out whether they have DNA sequences that are similar to opd.

TABLE 6
Bacteria Strains Possessing OPA Anhydrase Activity

| Strain | DFPase(mM/min) | Paraoxonase(mM/min |
|---|---|---|
| B. subtilus | 0.075 × 10⁻³ | 0.036 |
| globigii | | |
| P. acidovorans | ND | 0.059 |
| Flavobacterium sp. | 29.5 × 10⁻³ | 4.88 |
| ATCC 27551 | | |
| E. coli JM109 | 0.006 × 10⁻³ | 0.23 |
| E. coli JM103/opd | 0.05 × 10⁻³ | 0.42 |
| P. diminuta PD3⁺ | 61.7 × 10⁻³ | 1.34 |

reaction conditions: 50 mM BTP pH 7.2, 400 mM KCl, 50 mM NaCl, 100 um ZnCl2, 400 uM Mncl2,

The principle of the invention and the best mode contemplated for applying that principle have been described. It was to be understood that the foregoing was illustrative only and that other means and techniques can be employed without departing from the true scope of the invention defined in the following claims.

### BIBLIOGRAPHY

1. Munnecke, D. M. and Hsieh, D. P. H. 1976. Appl. Environ. Microbiol. 31: 63-69.
2. Sethunathan, N. and Yoshida, T. 1973. Can. J. Microbiol. 19: 873-875.
3. Munnecke, D. M. 1981 p. 251-270. In T. Leisinger, A. M. Cook, R. Hutter, and J. Nuesc (eds.), Microbial Degradation of Xenobiotics and Recalcitrant Compounds. Academic Press, Inc. London.
4. Serdar, C. M., et al., 1982. Appl. Environ. Microbiol. 44: 246-249.
5. Mulbry, W. W., et al. 1986. Appl. Environ. Microbiol. 51: 926-930.
6. McDaniel, C. S. 1985. Ph.D. Dissertation. Texas A & M University.
7. Chaudry, G. R., et al. 1988. Appl. Environ. Microbiol. 54: 288-293.
8. Merritt, G. C., et al. 1981. Austral. Vet. J. 57: 531.
9. Serdar, C. M. and Gibson, D. T. 1985. Bio/Technology 3: 567-571.
10. Brown, K. A. 1980. Soil Boil. Biochem. 12: 105-112.
11. Chiang, T., et al. 1985. Bull. Environ. Contam. Toxicol. 34: 809-814.
12. Bolivar, F., et al. 1977. Gene 2: 95-113.
13. Messing, J., et al. 1977. Proc. Nat. Acad. Sci. USA 74: 3642-3646.
14. Munch-Petersen, A. and Neuhard, J. 1964. Biochim. BioPhys. Acta 80: 542-551.
15. Clewell, D. B. and Helinski, D. R. 1969. Proc. Nat. Acad. Sci. USA 62: 1159-1166.
16. Berns, K. I. and Thomas, C. A. 1965. J. Mol. Biol. 11: 476-490.
17. Casadaban, M. J., et al. 1980. J. Bacteriol. 143: 971-980.
18. Hewick, R. M., et al., 1981. J. Biol. Chem. 256: 7990-7996.
19. Frantz, B. and Chakrabarty, A. M. 1987. Proc. Nat. Acad. Sci. USA 84: 4460-4464.
20. Maniatis, T., et al. 1982. Molecular Cloning: A Laboratory Manual. Cold Spring Harbor Laboratory. Cold Spring Harbor, N.Y.
21. Luckow, V. A. and Summers, M. D. 1988. Virology 167: 56-71.
22. Vaughn, J. L., et al. 1977. In Vitro 13: 213-217.
23. Hink, W. F. 1970. Nature 226: 466-470.
24. Smith, P. K., et al. 1985. Anal. Biochem. 150: 76-85.
25. Laemmli, U. K. 1970. Nature 227: 680-685.
26. Wray, W., et al. 1981. Anal. Biochem. 118: 197-203.
27. Scores, N. F. A. and Lichenstein, E. P. 1967. Econ. Entomol. 60: 1539-1544.
28. Struck, D. K., et al. 1985. J. Mol. Appl. Genet. 3: 18-25.
29. Southern, E. M. 1975. J. Mol. Biol. 98: 503-517.

1.-72. (canceled)

73. An isolated nucleic acid molecule comprising:
a nucleotide sequence of SEQ ID NO:3; and
a coding sequence, encoding an organophosphorous acid anhydrase.

74. (canceled)

75. The isolated nucleic acid molecule of claim 73, wherein the isolated nucleic acid molecule is a plasmid.

76. The isolated nucleic acid molecule of claim 73, wherein the isolated nucleic acid molecule is a viral vector.

77. The isolated nucleic acid molecule of claim 73, wherein the isolated nucleic acid molecule is an expression vector.

78. The isolated nucleic acid molecule of claim 77, wherein the expression vector expresses an organophosphorous anhydrase enzyme in a bacterial cell.

79. The isolated nucleic acid molecule of claim 77, wherein the expression vector expresses an organophosphorous anhydrase enzyme in an isolated eukaryotic cell.

80. A bacterial cell transformed with the expression vector of claim 78.

81. An isolated eukaryotic cell that transformed with the ession vector of claim 79.

82. The isolated eukaryotic cell of claim 81, further defined as a mammalian cell.


SEQUENCE LISTING (1 sequence)

SEQ ID NO: 1 - protein, 337 aa, Pseudomonas aeruginosa
MSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDRVNPDGMAFIPLRVIPFLREKGVPQETLAGITVTNPARFLSPTLRAS